(19) United States
(12) Patent Application Publication
(10) Pub. No.: US 2015/0272620 A1
(43) Pub. Date: Oct. 1, 2015

(54) Methods and Devices for Removing Tissue During a Female Patient's Laparoscopic Surgery

(71) Applicant: Zisow Surgical, LLC, Baltimore, MD (US)

(72) Inventor: David L. Zisow, Baltimore, MD (US)

(73) Assignee: Zisow Surgical, LLC, Baltimore, MD (US)

(21) Appl. No.: 14/249,406

(22) Filed: Apr. 10, 2014

Related U.S. Application Data
(63) Continuation-in-part of application No. 14/231,822, filed on Apr. 1, 2014.

(51) Int. Cl. A61B 17/42; A61B 17/32; A61B 17/3205; A61B 17/02

(52) U.S. Cl. CPC A61B 17/42; A61B 17/0293; A61B 17/32002; A61B 17/3205; A61B 2017/320064; A61B 2017/320024

(57) ABSTRACT

An improved system for isolating and removing a large mass of tissue from a female patient's abdominal cavity during a laparoscopic surgery, includes: (1) a wound retractor platform, (2) a vaginally-insertable, tissue collection bag having a flexible and resilient ring at its open end that is distortable for vaginal insertion but quickly resumes its open and undistorted shaped within the patient's abdominal cavity upon insertion, and whose open end receives the large mass of tissue and is then connected to the wound retractor platform so as to form a sealed bag, (3) a tissue cutting instrument which cuts the large mass of tissue into smaller pieces and then aspirates them along with some of the surrounding fluid, and (4) a fluid recirculation system which filters and recirculates the aspirated fluid back through the bag.

### CROSS-REFERENCE TO RELATED APPLICATIONS

This a Continuation-In-Part Patent Application and claims the benefit of U.S. patent application Ser. No. 14/231,822, filed Apr. 1, 2014 by the present inventor. The teachings of this application are incorporated herein by reference to the extent that they do not conflict with the teaching herein.

### BACKGROUND OF THE INVENTION

1. Field of the Invention The present invention generally relates to surgical devices and methods for collecting large mass tissue specimens surgically removed from a female patient. More specifically, the invention is directed to methods and devices for removing a large mass of tissue through a small incision in a female patient's abdominal cavity during a laparoscopic surgical procedure like a hysterectomy.

2. Description of the Related Art

Laparoscopic surgery in a patient's abdominal cavity is typically performed through devices known as wound retractor platforms, which: (a) protect a surgical incision into the wall of the abdominal cavity while also enhancing the exposure through it, (b) seal the abdominal cavity, (c) and permit the mounting and introduction into the cavity of surgical instruments, etc. through relatively small openings or access ports in these devices. This sealing is necessary because of the need to increase and maintain the size of the workable space around the operative site within the abdominal cavity, and the consequent use of insufflation gases which are introduced to inflate the cavity and elevate the abdominal wall. The pressurizing of the abdominal cavity is referred to as pneumoperitoneum. See, for example, U.S. Pat. Nos. 7,704,207, 7,815,567, 7,883,461 and 7,909,760.

In these surgeries, tissue disposed within the abdominal cavity is typically excised and removed from the body. However, the removal of such tissue often proves to be difficult for many reasons, including the limited confines or working is spaces inherent in such laparoscopic surgeries and the relatively small openings or access ports through wound retractor platforms.

Also, such excised tissue may include an infected or cancerous mass or organ, as well as blood, bile and other liquids, all referred to herein as tissue, which may pose infection issues or other complications if it is not enclosed while it is being transported from the surgical site to outside the abdominal cavity. Thus, it is always desirable to enclose such tissue as soon as possible at the surgical site and to keep it enclosed during its transport out of the abdominal cavity.

A quick survey of the patent literature in this area reveals that it is replete with myriad and diverse tissue retrieval and enclosure devices. Many of these utilize relatively small, rolled-up or folded bags or pouches that are deployed and opened in the abdominal cavity where tissue is placed in them and then they are closed for retraction. See, for example, U.S. Patent Publications 8,652,147, 8,486,087, 8,409,112, 7,650,887, 6,409,733, 5,647,372, 2009/0043315, 2009/0192510 and 2008/0221588.

A hysterectomy is the surgical removal of the uterus and is the second most common type of major surgery performed on women of childbearing age. A myomectomy is another commonly performed gynecologic procedure to remove fibroids while preserving the uterus. Advances in laparoscopic surgical equipment have enabled surgeons to remove fibroids and/or the uterus and/or ovaries laparoscopically. However, because of the large mass of such structures, these surgeries can present significant tissue enclosure and removal challenges.

The surgeon in a laparoscopic hysterectomy or myomectomy will usually have to expend a considerable amount of time and effort dissecting the fibroid(s) or uterus to small enough pieces so that they can be removed through the relatively small size of the opening of a trocar or other abdominal incision site. To help with this task, the surgeon may use a surgical instrument known as a morcellator, which consists of a hollow cylinder that penetrates the abdominal wall and has, at its open end, sharp edges that are used, often with a grasper that pulls the tissue into the cylinder, to mechanically cut or pulverized tissue.

A concern has arisen that the methods and instruments currently being used in laparoscopic surgeries are not doing a sufficiently adequate job in isolating and enclosing, during its removal from the abdominal cavity, the large mass of tissue involved in such surgeries. Cases have been alleged wherein, during a laparoscopic procedure, a previously undiagnosed sarcoma has been, because it was not adequately enclosed after excision and during removal, seeded throughout the abdominal cavity and thereby worsening a patient's prognosis.

To minimize this risk, what are needed are new laparoscopic surgery methods devices, and systems for better enclosing and removing the large masses of tissues that typically need to be removed in gynecologic surgery. The present invention seeks to provide such improved methods, devices and systems.

### SUMMARY OF THE INVENTION

Recognizing the need for improved methods, devices and systems for removing the large masses of tissue that typically need to be removed in the laparoscopic surgeries of female patients, the present invention is generally directed to providing such improved methods, devices and systems.

In a preferred embodiment, the present invention is a system for better isolating and removing a large mass of tissue from a female patient's abdominal cavity during a laparoscopic surgery. This improved system includes: (1) a wound retractor platform with ports through it that provide access to a patient's abdominal cavity when the wound refractor platform is being used to retract and enclose a laparoscopically-appropriate incision through the patient's abdominal wall, (2) a vaginally-insertable, tissue collection bag having a flexible and resilient ring that is attached to the open end of a transparent, flexible sleeve that has an interior volume which is sized so as to allow the large tissue mass to reside within the bag, (3) a tissue cutting instrument having a configuration adapted to: (3-i) extend the distal end of its elongated, hollow tubular member, which has within it a cutting member, though the wound retractor platform and into the bag in which resides the large tissue mass so that its cutting member can engage the tissue and cut it into smaller pieces, (3-ii) through an aspiration port in its hollow, tubular member, aspirate the smaller tissue pieces from the bag along with some of the fluid that surrounds them, and (4) a fluid recirculation system having a configuration adapted to filter and recirculate the aspirated fluid back through the bag so as to distend it to provide a laparoscopic surgeon with the largest possible working area and the greatest possible visibility in the bag.

In a first variant of this preferred embodiment, its wound retractor platform has: (a) a flexible and resilient, outer loop having an annular axis and adapted for juxtaposition with the outer surface of the abdominal wall, (b) a flexible and resilient, inner loop adapted for juxtaposition with the inner surface of the abdominal wall, (c) a membrane coupling these loops which has a configuration adapted to traverse a laparoscopic-surgery-appropriate incision in the abdominal wall and to cooperate with the outer loop that can roll over itself around its annular axis so as to roll this membrane around the outer loop and thereby retract and enclose the abdominal wall's incision, and (d) a cap having a configuration adapted for coupling the cap to the outer loop so as to provide the platform with the ability to seal the abdominal incision and cavity during a laparoscopic surgery; the cap also having a port that provides a sealed access through the cap and into the abdominal cavity and a removable portion that has a surrounding edge, with this removable portion being resealable so as to provide a second, larger pathway through the cap for gaining access to a surgical site.

In a second variant of this preferred embodiment, the configuration of its tissue collection bag is further adapted so that: (a) after its ring has been sufficiently distorted so as to allow it to be vaginally inserted to gain access into the abdominal cavity of the patient, and upon being situated in the abdominal cavity, the ring will return to essentially the shape that it took prior to being deformed for its vaginal insertion, and (aii) its ring can be further distorted so that it can be removed from the patient's abdominal cavity through the wound retractor platform when its cap is removed, and (b) its sleeve has a configuration that enables a portion of the sleeve that is proximate its open end to be attached, when the removable portion of the wound retractor platform cap has been removed, to the edge of the removable portion of the wound retractor platform's cap and to cooperate with the removable portion, once it has been again attached to its cap, so as to form, with the portion of the collection bag's sleeve that then remains in the patient's abdominal cavity and the wound retractor platform, a sealed bag that is accessible though the port in the wound retractor platform's cap.

In a third variant of this preferred embodiment, its fluid recirculation system includes: (a) a fluid reservoir that receives fluid from an external source, (b) a fluid output line that provides for the transport of fluid from the reservoir to the sealed bag, (c) a fluid return line that connects with the tissue cutting instrument's aspiration port and provides for the transport of fluid from the sealed bag to the reservoir, (d) a filter in the system that has a configuration adapted to filter the tissue from the fluid coming from the sealed bag so as to maintain the surgeon's visibility while working within the sealed bag, (e) one or more pumps in the system that recirculate the fluid through the system, and (f) a control system that monitors and controls the recirculation of the fluid through the sealed bag so as to maintain a desired level of distention of the sealed bag and sufficient cleanliness of the fluid so as to help provide a laparoscopic surgeon with a required level of visibility in the sealed bag.

In a fifth variant of this preferred embodiment, its fluid circulation system may also include a fluid make-up mechanism configured to monitor the fluid leaving the sealed bag and add additional fluid as necessary to the system so as to compensate for any fluid loss in the tissue filtering step.

In a sixth variant of this preferred embodiment, it may also include: (5) an introducer instrument or laparoscope having a configuration adapted to: (5-i) enable extending the distal end of the laparoscope though the wound retractor platform and to the surgical site, (5-ii) illuminate and view the surgical site, (2-iii) through its instrument port, allow other instruments, for example, the tissue cutting member, to pass through it to access the surgical site, and (2-iv) through its fluid introduction port, allow fluid to pass through it and into the surgical site.

Thus, there has been summarized above (rather broadly and understanding that there are other preferred embodiments which have not been summarized above) the present invention in order that the detailed description that follows may be better understood and appreciated.

### BRIEF DESCRIPTION OF THE DRAWINGS

FIG. 1 is a perspective illustration of a preferred embodiment of the present invention.

FIG. 2 is an illustrative depiction that shows the tissue collection bag of the present invention having been vaginally inserted into a female patient's abdominal cavity and at a point in time during a laparoscopic surgery when the bag is being used to isolate and enclose a large mass of excised uterine tissue.

FIG. 3 is an illustrative depiction that shows the tissue collection bag of the present invention having been vaginally inserted into a female patient's abdominal cavity and at a point in time during a laparoscopic surgery when the ring of the bag has been removed from the abdominal cavity but the bottom of the collection bag is still within the abdominal cavity and encloses a large mass of excised uterine tissue.

FIG. 4 shows a perspective view of a preferred embodiment of the tissue collection bag of the present invention.

FIG. 5 shows a perspective view of a preferred embodiment of the wound retractor platform of the present invention as it is situated in a patient's abdominal wall and before the tissue collection bag has been attached and sealed to the platform.

FIG. 6 provides an illustrative depiction of a point in time during a laparoscopic surgery that is after the excised large uterine mass has been enclosed in the tissue collection bag and after the ring of the bag has been extracted from the abdominal cavity through the removable portion of the wound retractor platform's cap.

FIG. 6A is an exploded view of that portion of FIG. 6 that shows the distal end of the tissue cutting instrument and its cutting window and cutting member.

### DESCRIPTION OF THE PREFERRED EMBODIMENT

Before explaining at least one embodiment of the present invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The present invention generally relates to tissue isolation and removal methods, devices and systems that can better isolate an excised, large uterine mass 1 during its removal from the abdominal cavity during a laparoscopic surgery. Since the surgical procedures involved in a laparoscopic surgery are well known in the art, they will not be discussed in detail herein. Instead, only the novel steps and devices is of the present invention and how they differ from those utilized in a conventional laparoscopic surgery will be discussed in detail.

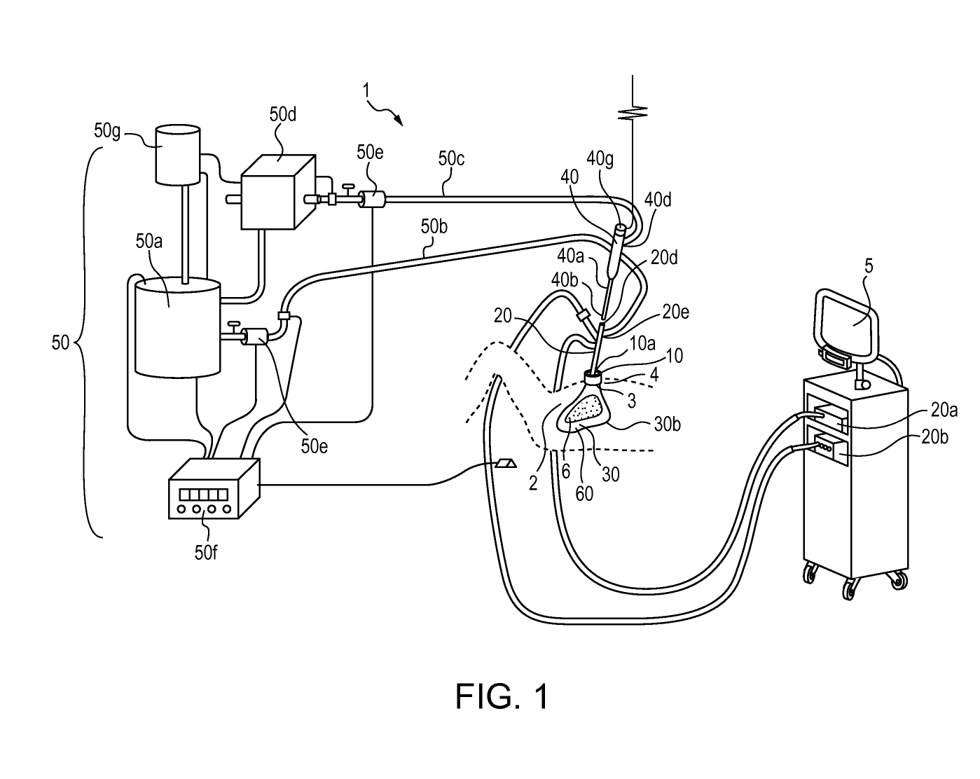

FIG. 1 provides a perspective illustration of an expanded embodiment of the present invention 1. Shown in FIG. 1 is a wound refractor platform 10 with ports through it that provide access to a patient's abdominal cavity 2 when the wound retractor platform is being used to retract, enclose and protect a laparoscopically-appropriate incision 3 through the patient's abdominal wall 4.

An introducer instrument or laparoscope 20 is used by the surgeon to gain access to the surgical site within the abdominal cavity, while also illuminating with its light source 20a and providing via its camera 20b a video image of the surgical site that can be displayed on a nearby monitor 5. Its distal end 20a extends through the wound retractor platform and into the abdominal cavity. It also has instrument 20d and fluid introduction 20e ports.

A tissue collection bag 30 is used to enclose and isolate the excised tissue 6 that is to be removed. This bag has a flexible and resilient ring 30a that is attached to the open end 30c of a transparent, flexible sleeve 30b that has an interior volume which is sized so as to allow the large tissue mass 6 to reside within the bag.

Also shown in FIG. 1 is a tissue cutting instrument 40 that is used to cut the excised tissue into smaller pieces that can more easily be removed from the bag. The distal end 40b of this instrument's elongated, hollow tubular member 40a is extended through the laparoscope's instrument port 20d or a port in the wound retraction platform and into the bag containing the excised tissue that is to be removed from the abdominal cavity. Within this member 40a is an electro-mechanical cutting member 40b that engages the tissue and cuts it into smaller pieces. An electric drive system 40g within the instrument provides the power for this cutting task. The instrument's hollow tubular member also has an aspiration port 40d through which the smaller tissue pieces are aspirated from the bag along, with some of the fluid that surrounds them.

A fluid recirculation system 50 is used to filter tissue from the aspirated fluid coming from cutting instrument 40 and clean it sufficiently so that it can be recirculated and used as the source of the fluid that is passed through the laparoscope 20 to distend either the abdominal cavity 6 or the collection bag 30. This fluid recirculation system generally includes: (a) a fluid reservoir 50a that initially receives fluid from an external source, (b) a fluid output line 50b that connects with the laparoscope's fluid introduction port 20d and provides for the transport of fluid from the reservoir to the bag, (c) a fluid return line 50c that connects with the tissue cutting instrument's aspiration port 40d and provides for the transport of fluid from the bag to the reservoir, (d) a filter 50d in the system that filters the tissue from the fluid coming from the bag and cleans it sufficiently so as to maintain the surgeon's visibility when this fluid is recirculated back into the bag to continue its distention, (e) one or more pumps 50e in the system that are used to aspirate the cutting instrument and recirculate the fluid through the system, and (f) a control system or programmable controller 50f that is used to monitor and control the recirculation of the fluid though the system so as to maintain a desired level of distention of the bag and sufficient cleanliness of the fluid so as to provide for adequate visibility within the bag. It may also include: (g) a fluid make-up mechanism 50g that is configured to monitor the fluid leaving the bag and add additional fluid as necessary to the system so as to compensate for any fluid loss in the tissue filtering step in order to maintain the desired distention level of the sealed bag.

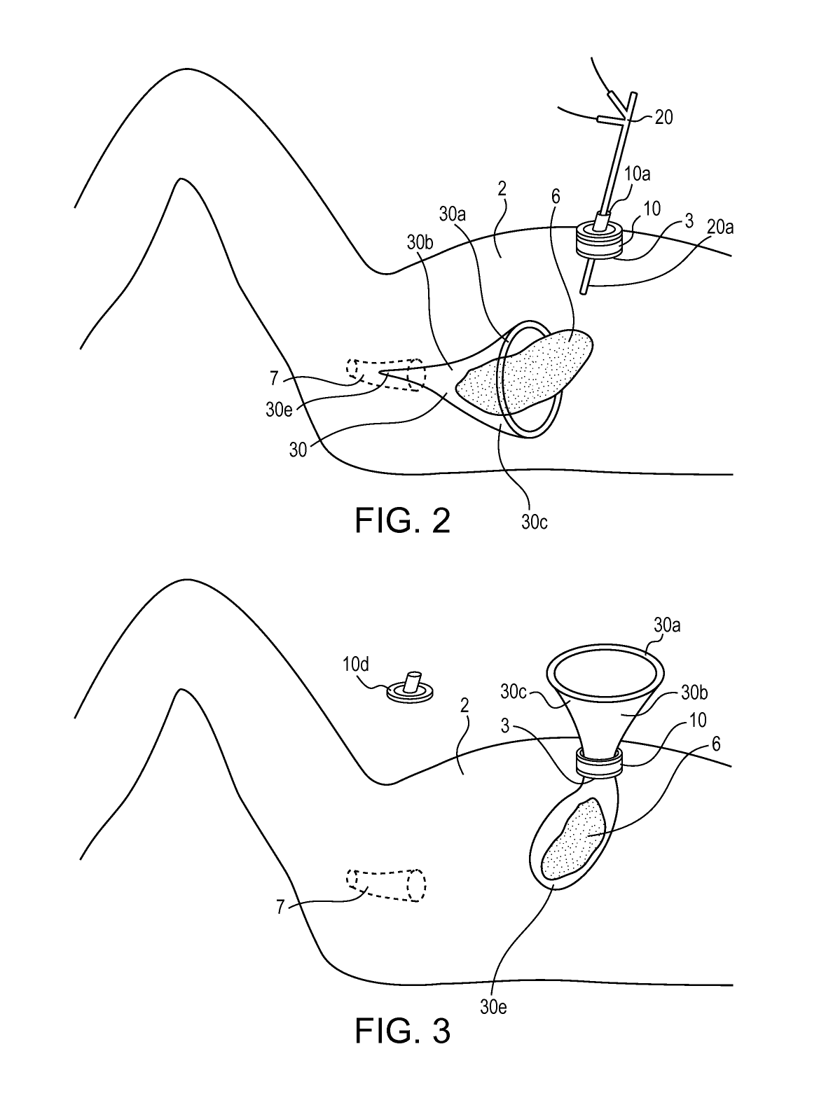

To better explain the method of the present invention, FIG. 2 provides an illustrative depiction of a point in time during a laparoscopic surgery that is soon after a uterine excision and before the site of the excision has been closed. An especially-designed, tissue collection bag 30, which has a flexible and resilient opening ring 30a, is seen to be introduced through the patient's vaginal canal 7 and into the abdominal cavity 2 where it is used to immediately enclose and isolate the large mass of tissue associated with the uterine excision. The resilience of the bag's ring allows it to quickly open and return to essentially the shape that it took prior to being deformed for its vaginal insertion.

In the case of total hysterectomy, the bag's insertion traverses the open vaginal cuff apex. In the case of a subtotal hysterectomy, a myomectomy, or excision of a large adnexal mass, the bag's insertion traverses an apical colpotomy incision into the pouch of Douglas.

This novel step in a laparoscopic surgery has many advantages. The bag's large, distortable and resilient opening ring 30a and its large enclosed, interior volume make it much easier for the surgeon to quickly get the large mass of excised tissue into this bag 30 and to therefore isolate it from the rest of the abdominal cavity. The surgeon is aided in this task by the fact that this large volume bag 30 is, due to its vaginal insertion, being pulled up from below the surgical site and the excised tissue. Consequently, a surgeon can get the excised tissue isolated quicker than the surgeon has ever been able to do so before using conventional laparoscopic surgery techniques.

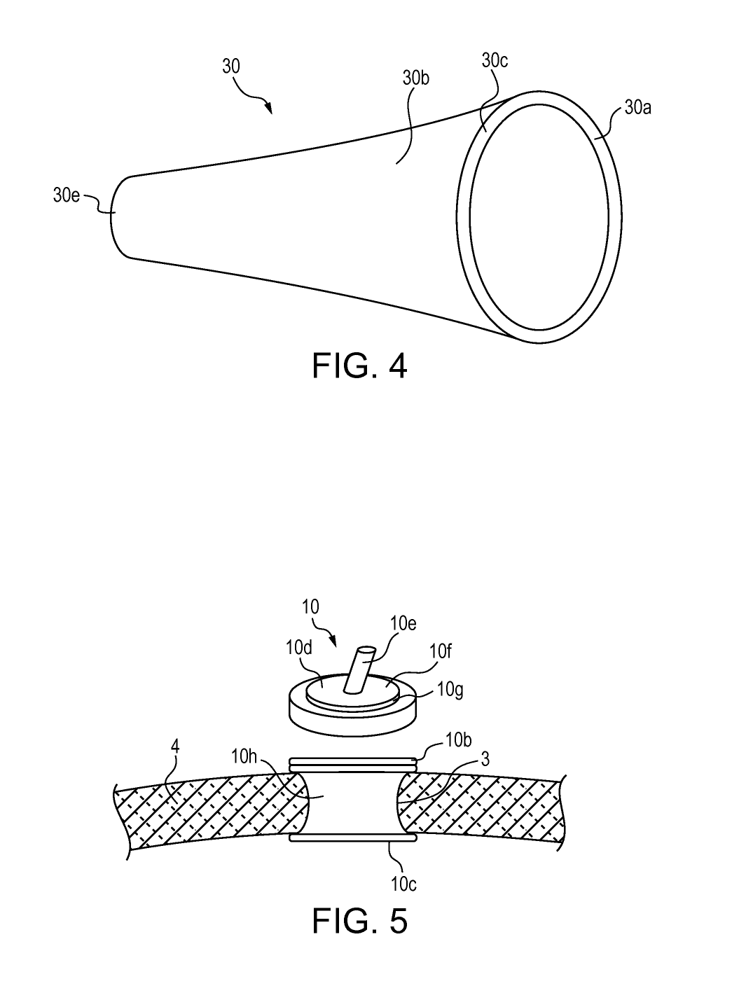

The configuration of this bag 30 have been adapted so that it allows any dissection or morcellation necessary for transport of the large mass out of the body to be performed within the isolated, large interior volume of the bag. The preparation for such processes is shown in FIGS. 3-4.

FIG. 3 shows an illustrative depiction of a point in time of a laparoscopic surgery during which the large area, opening ring 12 of the bag of the present invention has been distorted so that it could be removed from the patient's abdominal cavity 2 through the wound retractor platform 10 during a period of time when its cap 10d had been removed. This step in the method of the present invention has the benefit of enabling the excised uterine tissue to now be quickly and totally isolated by its enveloping bag 30 before any dissection or morcellation of this large mass is initiated.

FIG. 4 shows a perspective view of a preferred embodiment of the tissue collection bag 30 of the present invention. It is seen to include a flexible and resilient ring 30a that is deformable in order to enable it to be passed both through a patient's vaginal canal and a laparoscopic surgery incision in the patient's abdominal wall. This ring is initially released from its deformed shaped after it has passed through the uterine excision site and has reached the abdominal cavity. The overall shape of the ring can be circular, oval, elliptical or otherwise shaped to provide easy insertion through the vaginal canal and removal through a laparoscopic surgery incision or opening in the abdominal wall. Likewise, the cross-sectional shape through the ring 30a can be of any particular shape (e.g., round, elliptical, square, rectangular), and its materials of construction are selected so as to enhance the ring's ability to be flexible, resilient and durable.

A flexible walled, sleeve 30b made from a thin, tough, transparent, tissue-and bodily-fluids-impenetrable material (e.g., vinyl) includes an open proximal end 30c that is attached to the perimeter or circumference of the ring 30a and a closed distal end 30e. There are many ways known in the art to attach the open end of the sleeve to the ring and therefore these will not be discussed herein. The sleeve has an area of its open, proximal end and a length between its ends that provides the bag 30 with an interior volume that is sized so as to accommodate the expected large mass of excised tissue.

Special care should be taken in selecting the material of construction for this bag as its material must be capable of continuing to enclose and isolate the excised tissue despite the stresses imposed on it by reducing within the bag the tissue into smaller parts that can pass through the typical, small-sized, laparoscopic surgery incisions.

For a typical laparoscopic surgery, a preferred embodiment of this bag may have dimensions in the range of: a ring diameter=15-23 cm, the area of a cross-sectional section of the ring=0.3-1 cm², the length of the bag=25-40 cm, and the volume of the bag=1,500-10,000 cm³. Bags with dimensions in these ranges can easily isolate and contain tissue masses in the range of 500-2,000 gms and larger.

FIG. 5 shows an exploded view of a preferred embodiment of the wound retractor platform 10 of the present invention as it is situated in a patient's abdominal wall 4 and before the tissue collection bag 30 has been attached and sealed to it. Its purpose is to provide access to a patient's abdominal cavity 2 when the wound retractor platform 10 is being used to retract, enclose and protect a laparoscopically-appropriate incision 3 through the patient's abdominal wall 4. This platform has: (a) an outer ring or loop 10b that has an annular axis, with the loop being adapted for juxtaposition with the outer surface of the abdominal wall, (b) an inner ring or loop 10c adapted for juxtaposition with the inner surface of the abdominal wall, (c) a membrane 10h coupling these loops, with the membrane having a configuration adapted to provide it a neutral state and a transitional state, and in the neutral state, the outer loop 10b is substantially noncompliant, and in the transitional state, the outer loop rolls over itself around its annular axis to roll the membrane around the outer loop to retract and enclose the incision in the abdominal wall, and (d) a cap 10d having a configuration adapted for coupling the cap to the outer loop 10b so as to provide the wound retractor platform with the ability to seal the abdominal incision and cavity during a laparoscopic surgery. This cap also has a first aperture or port 10e that provides a sealed access through the cap and into the abdominal cavity and a removable portion 10f that has a surrounding edge 10g and is removable and resealable so as to provide a second pathway through said cap for gaining access to the abdominal cavity.

Such wound retractor platforms are well know in the surgical community and, therefore, more detailed information regarding this part of the present invention will not be included herein. For additional information on these platforms, see U.S. Pat. Nos. 7,704,207, 7,815,567, 7,883,461, 7,892,172 and 7,909,760.

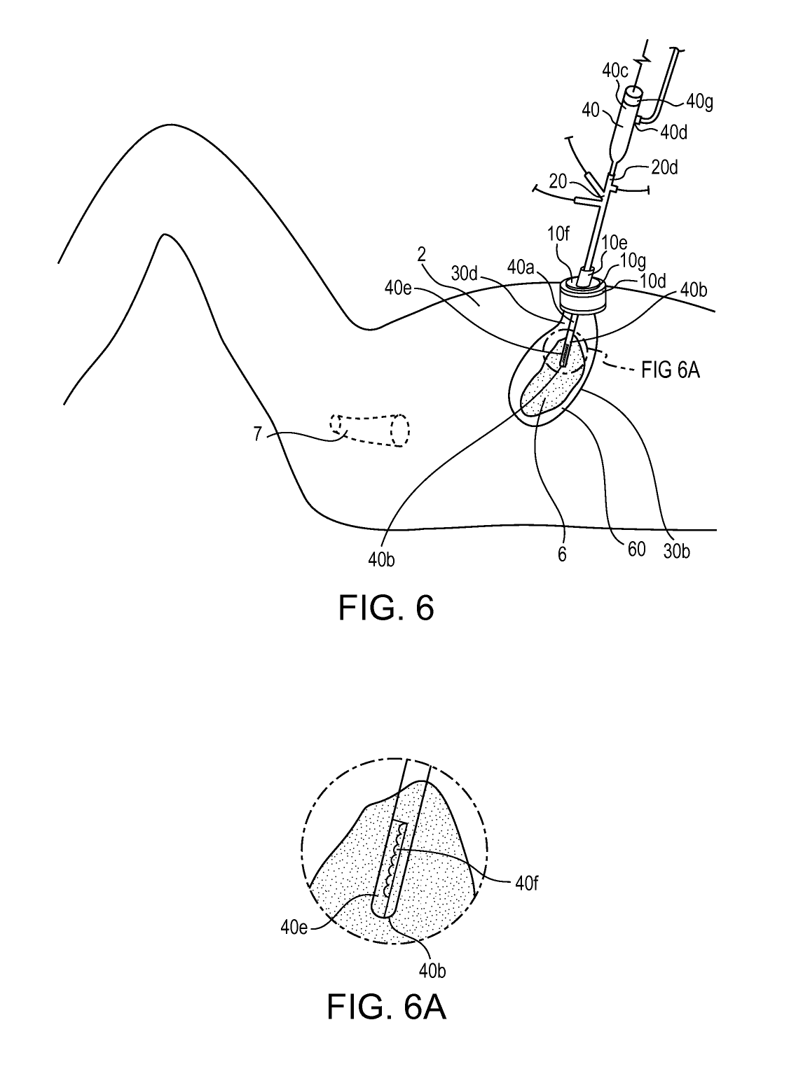

FIG. 6 provides an illustrative depiction of a point in time during a laparoscopic surgery that is after the excised large uterine mass 6 has been enclosed in the tissue collection bag 30 and after the ring 30a of the bag has been extracted from the abdominal cavity 2 through the removable portion 10f of the wound retractor platform's cap 10d. The sleeve 30b of this bag and the cap and its edge 10g have been especially configured so as to enable a portion 30d of the sleeve that is proximate the bag's open end to be attached, when the removable portion of the wound retractor platform cap has been removed, to the edge 10g of the removable portion of the wound retractor platform's cap and to cooperate with the removable portion 10f , once it has been again attached to its cap, so as to form, with the portion of the collection bag's sleeve that then remains in the patient's abdominal cavity and the wound retractor platform 10, a sealed bag 60 that is accessible, in this instance, though the port 10e in the wound retractor platform's cap. As shown here, that part of the collection bag that is above this sealed portion and the bag's ring 30a have optionally been removed so as to free up more room above the incision site. Also shown here is the distal end 40b of the tissue cutting instrument 40 having been passed through the laparoscope's instrument port 20d so as to place it proximate to the tissue 6 which is to be reduced in size when its comes proximate the instrument's cutting window 40e and its cutting member 40f, see FIG. 6A; thereby allowing these resulting smaller tissue parts to be removed through the tissue cutting instrument's aspiration port 40d.

The introducer instrument or laparoscope 20 that may be used as part of the present invention is well know in the art and, therefore, will not be described in great detail herein. It includes an elongated hollow tubular member who distal end 20c is provided with the ability to illuminate and capture a video image of the region in front of its distal end. It also has an instrument port 20d that allows other surgical instruments to pass through it to access the abdominal cavity, and a fluid introduction port 20e that allow fluid to pass through it for abdominal cavity distension or other purposes.

Fluid recirculation systems such as that which is part of the present invention are also well know in the art. See, for example, U.S. Pat. Nos. 8,596,118 and 8,626,311. Consequently, it will also not be described in great detail herein. Because of the large size of the tissue masses to be handed by the present invention and the desire to minimize a patent's time on the operating table, the cutting instruments of the present are quite robust and require a significant amount of aspiration to allow these large tissue masses to he quickly handled, etc. Such significant aspiration fluid flows make it advisable for the present invention to utilize a fluid cleaning and recirculation system. Additionally, this fluid can also be used to distend the sealed bag so as to provide a laparoscopic surgeon with the largest possible working area and maximum visibility around a surgical site.

The fluid recirculation system 50 of the present invention has: (a) a fluid reservoir 50a that receives fluid from an external source, (b) a fluid output line 50b that connects to an instrument, either the fluid introduction port 20e of the laparoscope or a similar port in a tissue cutting instrument 30 that is especially designed to provide both fluid outflow and aspiration, and whose distal end extends through the wound retractor platform 10 and provides for the transport of fluid from the reservoir 50a to the sealed bag 60, (c) a fluid return line 50c that connects to the cutting instrument's aspiration port 40d and provides for the transport of fluid from the sealed bag to the reservoir, (d) a filter 50d in the system that has a configuration adapted to filter tissue from the fluid coming from the sealed bag 60 so that this fluid can be recirculated without being so contaminated that its continued use would impair a surgeon's visibility while working within the sealed bag, (e) one or more pumps 50e in the system to aspirate the tissue cutting instrument and recirculate the fluid within the system, and (f) a control system 50f that monitors and controls the recirculation of the fluid though the sealed bag so as to maintain a desired level of distention of the sealed bag 60 and the cleanliness of the fluid within it so as to help provide a laparoscopic surgeon with a specified level of visibility in the sealed bag. Depending on the efficiency of this system's filter, it may also be necessary to utilize a fluid make-up mechanism 50g that is configured to monitor the fluid leaving the sealed bag 60 and add additional fluid as necessary to the system so as to compensate for any fluid loss in the tissue filtering step in order to help maintain a desired distention level of the sealed bag.

The technology for the tissue cutting instrument of the present invention is also well known in the art. See, for example, U.S. Pat. Nos. 5,602,449, 7,226,459 and 8,574,253. Such cutting instruments typically include an elongated, hollow, outer tubular member 40a having distal 40b and proximal 40c ends, an axial length therebetween, a cutting window 40e located proximate the distal end of this tubular member that provides an opening to its hollow interior, and an aspiration opening or port 40d proximate the proximal end of this member that provides an opening to its hollow interior and a passageway by which the smaller pieces of the cut tissue can be aspirated through the instrument. The axial length of this tubular member is such that during the instrument's use the proximal end 40c can reside outside the abdominal cavity 2 while the distal end 40b is passed through the wound retractor platform until it comes to rest proximate the excised tissue within the sealed bag.

A cutting member 40f is situated within the tubular member 40a and has a cutting end that is proximate a cutting window 40e which is located adjacent the member's distal end 40b. An electrically powered drive is coupled to the cutting member and causes the cutting end of the drive member to move and cooperate with the cutting window so as to cut into smaller pieces and tissue that is proximate the cutting window. Meanwhile, an aspiration opening 40d is located near the proximal end 40c of this member and is used to connect this instrument to the fluid circulation system 50 that provides the suction necessary to suck the cut pieces of tissue and some of the fluid that surrounds them from the seal bag 60 and through the instrument 40.

While the present invention has been described heretofore as an improved system for isolating and removing a large mass of tissue from the abdominal cavity of a female patient during a laparoscopic surgery, it should also be noted that the present invention also takes the form of the method for utilizing the specialized components or elements of this system. For examples, the methods of the present invention include such steps as:

(a) vaginally inserting the previously described tissue collection into the patient's abdominal cavity,

(b) enclosing and isolating the large excised mass within this collection bag,

(c) bringing this collection bag's ring through the wound retractor platform,

(d) utilizing the wound retractor platform's cap to clamp down upon a portion of the bag that is proximate its open end so as to seal it to the wound retractor platform so that it forms with that part of the collection bag which remains in the abdominal cavity a sealed bag which encloses and isolates the large tissue mass,

(e) utilizing the fluid recirculation system to distend the sealed bag,

(f) utilizing the tissue cutting member or morcellator to dissect or morcellate the large tissue mass into smaller pieces,

(g) utilizing the tissue cutting member's aspiration port and the fluid recirculation system to aspirate these smaller tissue pieces from the sealed bag, and

(h) when a sufficient volume of tissue has been aspirated from the bag, open the cap on the wound retractor platform and remove the sealed bag and its remaining contents from the abdominal cavity.

The foregoing is considered as illustrative only of the principles of the present invention. Further, since numerous modifications and changes will readily occur to those skilled in the art, it is not desired to limit the invention to the exact construction and operation shown and described herein. Accordingly, all suitable modifications and equivalents may be resorted to, falling within the scope of the invention that is hereafter set forth in the claims to the invention.

1. An improved system for isolating and removing at a surgical site a large mass of tissue from the abdominal cavity of a female patient during a laparoscopic surgery, said system comprising:
a tissue collection bag having: (a) a flexible and resilient ring with a circumference that encloses a specified ring open area, said ring having a configuration adapted to enable: (i) said ring to be distorted so that said entire ring can be vaginally inserted so as to gain access for said entire tissue collection bag into the abdominal cavity of said female patient so that said bag initially approaches said surgical site from below, and (ii) the sizing of said specified ring open area to be such that said large mass of tissue can pass though said ring open area, and (b) a sleeve having a flexible wall and open and closed ends between which said wall encloses and defines the interior volume of said sleeve, and wherein said sleeve open end is affixed to said ring, and said sleeve interior volume is sized so as to allow said large mass of tissue, after passing though said ring open area, to reside within and be isolated in said sleeve,
a wound retractor platform that retracts and encloses a laparoscopically-appropriately-sized incision through the abdominal wall of said patient and provides access to the abdominal cavity of said female patient and said surgical site, said wound retractor platform having: (a) a flexible and resilient, outer loop with an annular axis and adapted for juxtaposition with the outer surface of said patient abdominal wall, (b) a flexible and resilient, inner loop adapted for juxtaposition with the inner surface of said patient abdominal wall, (c) a membrane coupling said loops which has a configuration adapted to traverse said laparoscopic-surgery-appropriately-sized incision and to cooperate with said outer loop that is adapted to roll over itself around said annular axis so as to roll said membrane around said outer loop and thereby retract and enclose said incision in said abdominal wall, and (d) a removable cap having a configuration adapted for coupling said cap to said outer loop so as to provide the platform with the ability to seal said abdominal incision during a laparoscopic surgery; said cap also having a port that provides a sealed access through said cap and into said female patient abdominal cavity and a removable portion that has a surrounding edge, with said removable portion being resealable so as to provide a second, larger pathway through said cap for gaining access to said abdominal cavity and said surgical site of said female patient,
wherein said configuration of said tissue collection bag ring is further adapted so that: (a1) after said ring has been sufficiently distorted so as to allow said ring to be vaginally inserted to gain access into said patient abdominal cavity, and upon being situated in said abdominal cavity, said ring returns to essentially the shape that said ring took prior to being deformed for vaginal insertion, and (a2) said ring can be further distorted so that said entire ring and bag can be removed from the said female patient abdominal cavity through said laparoscopically-appropriately-sized, abdominal incision that said wound retractor platform is retracting and enclosing, and
wherein said sleeve having a configuration adapted to enable, once said sleeve has passed though said vaginal canal so as to initially approach said surgical site from below and said large mass of tissue has been placed into and isolated in said sleeve, a portion of the sleeve that is proximate said open end to be attached, when said removable portion of said wound retractor platform cap has been removed, to said edge of said removable portion of said cap and to cooperate with said removable portion, once said removable portion has been again attached to said cap, so as to form, with the portion of the sleeve of said collection bag that then remains in said female patient abdominal cavity and said wound retractor platform, a sealed bag that is accessible through a port in the cap of said wound retractor platform , and
wherein to provide for said vaginal insertion of said ring, said ring is dimensioned such that the cross-sectional section through said ring has a cross-sectional area in the range of 0.3-1 cm² and the circumference of said specified ring open area when said ring has assumed a circular shape is characterized by having an effective ring diameter in the range of 15-23 cm.

2. The system as recited in claim 1, said system further comprising:
a tissue cutting instrument having an elongated, hollow tubular member with a distal end, a cutting window and an aspiration port, and a cutting member situated in said tubular member, and a configuration adapted to: (i) extend said distal end through said wound retractor platform and into said sealed bag in which resides said large tissue mass so that said cutting member engages said tissue and dissects said tissue into smaller pieces, (ii) utilizing said aspiration port, aspirate said smaller tissue pieces from said sealed bag along with some of said fluid that surrounds said smaller tissue pieces.

3. The system as recited in claim 2, said system further comprising:
a fluid recirculation system having a configuration adapted to filter said smaller tissue pieces from said aspirated fluid and to recirculate said aspirated fluid back into said sealed bag.

4. The system as recited in claim 3, said system further comprising:
a laparoscope having a distal end and instrument and introduction ports and a configuration adapted to: (i) extend said distal end though said wound retractor platform, (ii) illuminate and view a surgical site, (iii) utilize said instrument port to allow the distal end of another surgical instrument to gain access to said surgical site, and (iv) utilize said fluid introduction port to allow fluid to gain access to and distend an enclosing cavity surrounding said surgical site.

5. The system as recited in claim 3, wherein:
said fluid recirculation system further having: (a) a fluid reservoir that receives fluid from an external source, (b) a fluid output line that connects with a port that provides access through said wound retractor platform so as to provide for the transport of fluid from said reservoir and into said sealed bag, (c) a fluid return line that connects with the aspiration port of said tissue cutting instrument to provide for the transport of fluid from the sealed bag to said reservoir, (d) a filter in said system that has a configuration adapted to filter the tissue from the fluid coming from said sealed bag, (e) a pump in said system that recirculates the fluid through said sealed bag, and (f) a control system that monitors and controls the recirculation of the fluid through said sealed bag.

6. The system as recited in claim 4, wherein:
said fluid recirculation system further having: (a) a fluid reservoir that receives fluid from an external source, (b) a fluid output line that connects with a port that provides access through said wound retractor platform so as to provide for the transport of fluid from said reservoir and into said sealed bag, (c) a fluid return line that connects with the aspiration port of said tissue cutting instrument to provide for the transport of fluid from the sealed bag to said reservoir, (d) a filter in said system that has a configuration adapted to filter the tissue from the fluid coming from said sealed bag, (e) a pump in said system that recirculates the fluid through said sealed bag, and (f) a control system that monitors and controls the recirculation of the fluid through said sealed bag.

7. The system as recited in claim 5, wherein:
said fluid circulation system further having a fluid make-up mechanism having a configuration adapted to monitor said recirculating fluid and add additional fluid as necessary so as to compensate for fluid loss in the tissue filtering step.

8. The system as recited in claim 6, wherein:
said fluid circulation system further having a fluid make-up mechanism having a configuration adapted to monitor said recirculating fluid and add additional fluid as necessary so as to compensate for fluid loss in the tissue filtering step.

9. An improved method for isolating and removing a large mass of tissue from the abdominal cavity of a female patient during a laparoscopic surgery, said method comprising the steps of:
utilizing a tissue collection bag having: (a) a flexible and resilient ring with a circumference that encloses a specified ring open area, said ring having a configuration adapted to enable: (i) said ring to be distorted so that said ring can be vaginally inserted so as to gain access into the abdominal cavity of said patient, and (ii) the sizing of said specified ring open area to be such that said large mass of tissue can pass though said ring open area, and (b) a sleeve having a flexible wall and open and closed ends between which said wall encloses and defines the interior volume of said sleeve, and wherein said sleeve open end is affixed to said ring, and said sleeve interior volume is sized so as to allow said large mass of tissue, after passing though said ring open area, to reside within and be isolated in said sleeve,
utilizing a wound retractor platform that retracts and encloses a laparoscopically-appropriate incision through the abdominal wall of said patient and
provides access to the abdominal cavity of said patient, said wound retractor platform having: (a) a flexible and resilient, outer loop with an annular axis and adapted for juxtaposition with the outer surface of said patient abdominal wall, (b) a flexible and resilient, inner loop adapted for juxtaposition with the inner surface of said patient abdominal wall, (c) a membrane coupling said loops which has a configuration adapted to traverse said laparoscopic-surgery-appropriate incision and to cooperate with said outer loop that is adapted to roll over itself around said annular axis so as to roll said membrane around said outer loop and thereby retract and enclose said incision in said abdominal wall, and (d) a removable cap having a configuration adapted for coupling said cap to said outer loop so as to provide the platform with the ability to seal said abdominal incision and cavity during a laparoscopic surgery; said cap also having a port that provides a sealed access through said cap and into said patient abdominal cavity and a removable portion that has a surrounding edge, with said removable portion being resealable so as to provide a second, larger pathway through said cap for gaining access to said abdominal cavity of said patient,
wherein said configuration of said tissue collection bag ring is further adapted so that: (a1) after said ring has been sufficiently distorted so as to allow said ring to be vaginally inserted to gain access into said patient abdominal cavity, and upon being situated in said abdominal cavity, said ring returns to essentially the shape that said ring took prior to being deformed for vaginal insertion, and (a2) said ring can be further distorted so that said ring can be removed from the said patient abdominal cavity through said wound retractor platform, and
wherein said sleeve having a configuration that enables a portion of the sleeve that is proximate said open end to be attached, when said removable portion of said wound retractor platform cap has been removed, to said edge of said removable portion of said cap and to cooperate with said removable portion, once said removable portion has been again attached to said cap, so as to form, with the portion of the sleeve of said collection bag that then remains in said patient abdominal cavity and said wound retractor platform, a sealed bag that is accessible through a port in the cap of said wound retractor platform.

10. The method as recited in claim 9, said method further comprising the step of:
utilizing a tissue cutting instrument having an elongated, hollow tubular member with a distal end, a cutting window and an aspiration port, and a cutting member situated in said tubular member, and a configuration adapted to: (i) extend said distal end through said wound retractor platform and into said sealed bag in which resides said large tissue mass so that said cutting member engages said tissue and dissects said tissue into smaller pieces, (ii) utilizing said aspiration port, aspirate said smaller tissue pieces from said sealed bag along with some of said fluid that surrounds said smaller tissue pieces.

11. The method as recited in claim 10, said method further comprising the step of:
utilizing a fluid recirculation system having a configuration adapted to filter said smaller tissue pieces from said aspirated fluid and to recirculate said aspirated fluid back into said sealed bag.

12. The method as recited in claim 11, said method further comprising the step of:
utilizing a laparoscope having a distal end and instrument and introduction ports and a configuration adapted to: (i) extend said distal end though said wound retractor platform, (ii) illuminate and view a surgical site, (iii) utilize said instrument port to allow the distal end of another surgical instrument to gain access to said surgical site, and (iv) utilize said fluid introduction port to allow fluid to gain access to and distend an enclosing cavity surrounding said surgical site.

13. The method as recited in claim 11, wherein:
said fluid recirculation system further having: (a) a fluid reservoir that receives fluid from an external source, (b) a fluid output line that connects with a port that provides access through said wound retractor platform so as to provide for the transport of fluid from said reservoir and into said sealed bag, (c) a fluid return line that connects with the aspiration port of said tissue cutting instrument to provide for the transport of fluid from the sealed bag to said reservoir, (d) a filter in said system that has a configuration adapted to filter the tissue from the fluid coming from said sealed bag, (e) a pump in said system that recirculates the fluid through said sealed bag, and (f) a control system that monitors and controls the recirculation of the fluid through said sealed bag.

14. The method as recited in claim 12, wherein:
said fluid recirculation system further having: (a) a fluid reservoir that receives fluid from an external source, (b) a fluid output line that connects with a port that provides access through said wound retractor platform so as to provide for the transport of fluid from said reservoir and into said sealed bag, (c) a fluid return line that connects with the aspiration port of said tissue cutting instrument to provide for the transport of fluid from the sealed bag to said reservoir, (d) a filter in said system that has a configuration adapted to filter the tissue from the fluid coming from said sealed bag, (e) a pump in said system that recirculates the fluid through said sealed bag, and (f) a control system that monitors and controls the recirculation of the fluid through said sealed bag.

15. The method as recited in claim 13, wherein:
said fluid circulation system further having a fluid make-up mechanism having a configuration adapted to monitor said recirculating fluid and add additional fluid as necessary so as to compensate for fluid loss in the tissue filtering step.

16. The method as recited in claim 14, wherein:
said fluid circulation system further having a fluid make-up mechanism having a configuration adapted to monitor said recirculating fluid and add additional fluid as necessary so as to compensate for fluid loss in the tissue filtering step.
